(19) United States
(12) Patent Application Publication
(10) Pub. No.: US 2012/0015846 A1
(43) Pub. Date: Jan. 19, 2012

(54) Label-free Cellular Pharmacology For Drug Antitarget Assessment

(76) Inventor: Ye Fang, Painted Post, NY (US)

(21) Appl. No.: 13/181,051

(22) Filed: Jul. 12, 2011

Related U.S. Application Data
(60) Provisional application No. 61/364,950, filed on Jul. 16, 2010.

(51) Int. Cl. C40B 30/06; C12Q 1/02

(52) U.S. Cl. 506/10; 435/29

(57) ABSTRACT

Described are methods relating to assessing antitargets of molecules. Also described are methods of screening molecules. In some aspects of the methods, the molecules are analyzed using a label free biosensor.

### CROSS-REFERENCE TO RELATED APPLICATIONS

This application claims the benefit of priority under 35 U.S.C. §119 of U.S. Provisional Application No. 61/364,950 filed on Jul. 16, 2010 the content of which is relied upon and incorporated herein by reference in its entirety.

### SEQUENCE LISTING

This application contains a Sequence Listing electronically submitted via EFS-Web to the United States Patent and Trademark Office as text filed named “20110712_SP10_{—}200_ST25.txt” having a size of 42,256 bytes and created on Jul. 12, 2011. Due to the electronic filing of the Sequence Listing, the electronically submitted Sequence Listing serves as both the paper copy required by 37 CFR §1.821(c) and the CRF required by §1.821(e). The information contained in the Sequence Listing is hereby incorporated herein by reference and does not go beyond the disclosure in the International Application as filed.

### BACKGROUND

Label free biosensor assays are desirable assays for monitoring cell activities because they allow for non-invasive and real time cellular analysis. Reliable, affordable and time effective drug antitarget assessment is crucial in drug development programs. Disclosed herein are methods related to drug antitarget assessment and screenings related to drug safety using label-free biosensors.

### SUMMARY

Described herein are methods, compositions, articles, and machines related to drug antitarget assessment. Also described herein are methods related to screening of candidate antitarget molecules. The methods described herein can be used with label-free biosensors.

### BRIEF DESCRIPTION OF THE FIGURES

FIG. 1 shows a schematic layout of a 96 well biosensor microplate having a panel of cells and cell types for drug antitarget assessment. 8 different cell types are cultured at the same time within a 96well biosensor microplate, and used for label-free antitarget profiling. The cell types share a common cellular background (e.g., HEK293 cell). The cell types include the parental HEK293 cells (negative control, row A), HEK-hERG (HEK cell stably expressing hERG1; row B), HEK-alpha1A (HEK cell stably expressing alpha1A adrenergic receptor; row C), HEK-D2 (HEK cell stably expressing dopamine D2 receptor; row D), HEK-5HT2A (HEK cell stably expressing 5-HT2A receptor; row E); HEK-5HT2C (HEK cell stably expressing 5HT2C receptor; row F), HEK-M1 (HEK cell stably expressing M1 receptor; row G), and HEK-5HT2B (HEK cell stably expressing 5HT2B receptor, row H). The cell types were sequentially stimulated with a drug test molecule at a specific dose (e.g., 10 micromolar), followed by a panel of markers, each for its respective receptor and at a specific concentration (e.g., EC50, EC80, 1×EC100, 2×EC100). The markers can be the protease activated receptor PAR1 agonist SFLLR-amide for endogenous PAR1 in the parental HEK293 cell, the hERG activator mallotoxin for HEK-hERG cell, the alpha1A adrenergic receptor agonist oxymetazoline or A61603 or epinephrine for HEK-alpha1A, the dopamine receptor agonist dopamine for HEK-D2, the 5HT agonist serotonin for HEK-5HT2A, HEK-5HT2B, and HEK-5HT2C, and the muscarinic receptor agonist carbachol or acetylcholine for HEK-M1.

### DETAILED DESCRIPTION

Drug development is a costly and time-consuming procedure. To reduce the risk of failure in late stages or after market introduction, it is preferred to investigate all potential side effects of a drug candidate as early as possible. Drugs typically exert their desired and undesired biological effects by virtue of binding interactions with protein target(s) and antitarget(s), respectively. An antitarget is a protein with which a drug molecule or a drug candidate molecule interacts, and which then results in adverse side effect(s) of the molecule in humans.

Traditional drug safety assessment commonly uses binding profiling and animal models. The binding profiling is not sufficient since it dose not provide information regarding the mode-of-action. The typical cellular assay is also not effective and accurate since they mostly measure one event downstream of receptor signaling. Animal models are costly and may not be predictive in humans.

Disclosed herein are methods, compositions, articles, and machines related to antitarget assessment for candidate antitarget molecules.

### A. hERG

One antitarget is the human ether-à-go-go-related gene (hERG) channel. hERG is a voltage gated ion channel, and is involved in regulating the movement of potassium ions across the cell plasma membrane. The hERG ion channel is a large tetramer protein. Besides playing a critical role in cardiac myocytes, increasing evidence has shown that hERG channel expression levels were elevated in several types of cancer cells including leukemia, colon cancer, gastric cancer, breast cancer and lung cancer cells.

Voltage-dependant ion channels are proteins that span cell surface membranes in excitable tissue such as heart and nerves. Ions passing through channels form the basis of the cardiac action potential. Influx of Na⁺ and Ca²⁺ ions, respectively, control the depolarizing upstroke and plateau phases of the action potential. K⁺ ion efflux repolarizes the cell membrane, terminates the action potential, and allows relaxation of the muscle. A rapid component of the repolarizing current flows through the K⁺ channel encoded by the human ether-a-go-go-related gene (hERG). Impaired repolarization can prolong the duration of the action potential, delay relaxation and promote disturbances of the heartbeat. Action potential prolongation is detected clinically as a lengthening of the QT interval measured on the electrocardiogram (ECG). Drug-induced QT prolongation is a serious complication of drugs due to impaired repolarization, which is associated with an increased risk of lethal ventricular arrhythmias. Drug-induced QT prolongation is almost always associated with block of the hERG K⁺ channel. A plethora of drugs, such as methanesulfonanilides, dofetilide, MK-499, and E-4031 are known to block K⁺ ion channels, such as hERG, on the heart causing a life threatening ventricular arrhythmia and heart attack in susceptible individuals. Unfortunately, incidence of drug-induced ventricular arrhythmia is often too low to be detected in clinical trials.

A sudden death due to the blocking of hERG channels by noncardiovascular drugs such as terfenadine (antihistamine), astemizole (antihistamine), and cisapride (gastrokinetic) led to their withdrawal from the market. Recently, drugs like Vioxx were also pulled out of the market for concerns relating to dangerous cardiac side effects. Consequently, cardiac safety relating to K⁺ channels has become a major concern of regulatory agencies. In order to prevent costly attrition, it has therefore become a high priority in drug discovery to screen out inhibitory activity on hERG channels in lead compounds as early as possible.

Current methods for testing potential drug molecules for hERG blocking activity have several limitations. Technologies based on cell-based patch clamp electrophysiology or animal tests are technically difficult and do not meet the demand for throughput and precision for preclinical cardiac safety tests. Other assays use radio-labeled, fluorescent, dye-conjugated, or biotinylated markers for detection and quantification of binding. However, many of these markers have reduced activity after labeling. In addition, the use of radio-labeled analogs poses practical limitations such as requirements for complex infrastructure and licenses for operating radioactive compounds. The promiscuous nature of this channel, referred to herein as the hERG K⁺ channel, or hERG, or hERG ion channel, or hERG channel, leads to it binding a diverse set of chemical structures (Cavalli, A., et al., J. Med. Chem. 2002, 45(18), 3844-53), coupled with the potential fatal outcome that may emerge from that interaction is a problem. These realities have resulted in the recommendation from the International Congress of Harmonization and the U.S. Food and Drug Administration that all new drug candidates undergo testing in a functional patch-clamp assay using the human hERG protein, either in native form or expressed in recombinant form (Bode, G., et al., Fundam. Clin. Pharmacol. 2002, 16(2), 105-18). Although automated, high-throughput patch-clamp methods have recently been developed, such systems require specialized operators, live cells, and a substantial capital investment (Bridgland-Taylor, M., et al., J. Pharmacol. Toxicol. Methods 2006, 54(2), 189-99; Dubin, A., et al., J. Biomol. Screen. 2005, 10(2), 168-81). Accordingly, there is a need to develop new compositions and methods for characterizing and quantifying the binding of molecules, such as drug candidates, to hERG channels.

Thus, hERG channel inhibition has been recognized as the main reason for severe, even fatal, cardiac side effects of many lipophilic compounds and drug candidates. The primary mode of action for the side effects is that the drug or drug candidate blocks the hERG channel which can lead to QT prolongation, which is a risk factor for torsades de pointes, a potentially life-threatening arrhythmia. A growing list of agents with “QT liability” have been withdrawn from the market or restricted in their use, whereas others did not even receive regulatory approval for this reason. Another prominent example is the non-sedative antihistaminic Terfenadine, which had to be withdrawn from the market because of such a side effect.

Another mode of action associated with hERG liability is the activation of hERG channel which leads to short QT syndrome. Several hERG channel activators have been identified so far, including RPR260243, NS1643, NS3623, PD-118057, PD-307243, mallotoxin and A-935142 (see Su, Z., et al. Electrophysiologic characterization of a novel hERG channel activator. Biochem Pharm 77:1383, 2009). These hERG activators have diverse chemical structures and enhance the hERG channel activity by different mechanisms. Among these known hERG activators, PD-118057, NS3623 and RPR260243 have been shown to shorten both the ventricular AP duration and the QT interval. RPR260243 and PD-118057 can reverse the AP prolonging effects of dofetilide. The mechanism of action of these channel activators is varied. NS1643 and NS3623 primarily reduce the inactivation of hERG by shifting its voltage dependence rightward; neither compound was designed to interact with the S5-pore linker, and their sites of action with the hERG channel are as yet unknown. Mallotoxin affects all three, strongly shifting the activation curve leftward, but also slowing deactivation and having minor effects on inactivation. In addition, it may be possible to modulate hERG activity with drugs acting on protein kinases, as hERG current can be modulated by protein kinase A and protein kinase C activity. Thus, hERG K⁺ channels have become a primary antitarget (i.e. an unwanted target) in drug development.

1. hERG Assays

Because of the importance of hERG channels in drug safety issues, various technologies have been developed for hERG functional assays. Traditional hERG ion channel assays typically involves assaying ion flux such as Rb⁺ flux using ion absorption assays, or assaying hERG currents directly using patch clamping methods. Electrophysiology manual patch clamp technique has always been regarded as the gold standard for hERG studies. However, manual patch clamping requires an experienced electrophysiologist and is very labor intensive. In recent years automated patch clamping technologies (such as IonWorks from Molecular Devices) have been developed which allow medium to high throughput testing of a compound's effect on hERG channel function. Different from patch clamping, which measures hERG activity from a single ruptured cell, fluorescent membrane potential assays or Rb⁺ flux assays measure the hERG channel response from a population of cells. In addition, biochemical assays have also been used for measuring compound binding affinity with hERG protein. However, lacking physiologically functional assay readouts, particularly with high throughput, is considered a bottleneck in drug discovery for ion channels.

Since label-free biosensor, particularly optical biosensors, cellular assays are largely sensitive to mass redistribution within its sensing volume or detection volume, it is commonly believed that these biosensor cellular assays are not amenable to monitoring ion channel activities directly.

Label-free cellular assays are largely considered to be non-specific, due to their integrative and generic nature of the biosensor readout or output signals obtained. A given biosensor output signal induced by a compound could be originated from different possible cellular processes or signaling events. Also, the possibility of a compound having polypharmacology, which is quite common to almost all compounds, complicates the assignment of modes of action of the compound observed with these biosensor cellular assays.

In certain label free cell assay methods, one has a cell line, a target, an activator (or modulator), and then a marker. These combinations can be used to assay for diverse arrays of modulators (See for example, WO2006108183 Fang, Y., et al. “Label-free biosensors and cells”).

Screening using a typical label free cell assay target approach (i.e., screening using a specific cell line expressing the target of interest, e.g., HEK-hERG cells) commonly leads to high false positives. This is because label-free cellular assays are often for profiling molecules at a pathway level due to the integrative nature of the biosensor signal of the target receptor (meaning that modulators that not only act on the target of interest, but also on the target-associated pathway(s) are detected together from a screen). As a result, screening using the typical label-free cell assay target approach leads to information about the pathways and targets involved in these, but often exhibits low specificity at the target level. The methods disclosed herein use the information that can be gained from label free target assays and label free pathway assays, to achieve screen results with higher specificity at the target level.

The disclosed methods provide a higher resolution of assessment of modulators acting on a specific target then in previous label free integrated pharmacology methods, such as those disclosed in U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009.

U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009, both of which are herein incorporated by reference at least for material related to label free assays and methods. In the methods disclosed in U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009, a panel of markers is chosen and assayed, and this information provides information about the pathways in the cell connected to the markers. The disclosed methods use identified cells, based on appropriate pathways for specific targets. In certain embodiments, the information used from methods disclosed in U.S. application Ser. No. 12/623,693. Fang, Y., Ferrie, A. M., Lahiri, J., and Tran, E. “Methods for Characterizing Molecules”, Filed Nov. 23, 2009 and U.S. application Ser. No. 12/623,708. Fang, Y., Ferrie, A. M., Lahiri, J., and Tran, E. “Methods of creating an index”, filed Nov. 23, 2009, can be used to provide the information and identified cells which can be used in the methods disclosed herein.

Also disclosed are assays related to the use of label free biosensors and hERG which identify hERG modulators. Information regarding this can be found in U.S. Patent Application No. 61/291,731, Fang, Y., Sun, H., “Label-free methods related to hERG potassium ion channels” which was filed Dec. 31, 2009.

### B. G PROTEIN-COUPLED RECEPTORS (GPCR)

Another class of antitargets is G protein-coupled receptors (GPCRs). GPCRs form a large protein family that plays an important role in many physiological and pathophysiological processes. GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Historically, the discovery of drugs acting at GPCRs has been extremely successful with 50% of all recently launched drugs targeting GPCRs. In particular, the subfamily of biogenic amine binding GPCRs has provided excellent drug targets for the treatment of numerous diseases, such as schizophrenia (mixed D2/D1/5-HT2 antagonists), psychosis (mixed D2/5-HT2A antagonists), depression (5-HT1 agonists), migraine (5-HT1 agonists), allergies (H1 antagonists), asthma (beta2 adrenergic receptor agonists, M1 antagonists), ulcers (H2 antagonist), and hypertension (beta1 adrenergic receptor antagonist). However, the central roles that many of the biogenic amine binding GPCRs play in cell signaling also poses a risk in new drug candidate molecules that reveal side affinities towards these receptor sites. These drug candidate molecules have the potential to interfere with the physiological signaling processes and to cause undesired effects in preclinical or clinical studies. For example, the alpha1A adrenergic receptor modulates the relaxation of the vascular muscle tone and is thus important for blood pressure regulation. Therefore, the alpha1A adrenergic receptor has been classified as an antitarget that mediates cardiovascular side effects of many drug candidates, causing orthostatic hypotension, dizziness, and fainting spells. Furthermore, in order to obtain a clean clinical profile for novel development drug candidate molecules, strong off-target molecular interactions with dopamine and serotonin receptors (like the 5-HT2A and D2 receptors), which represent the molecular targets for many antipsychotics (for example, olanzapine or risperidone), needs to be avoided.

To date, there are several GPCRs that have been classified as antitarget GPCRs. One antitarget is the alpha1A adrenergic receptor. The adrenergic alpha1A receptor is a key antitarget for cardiovascular side effect including orthostatic hypotension, dizziness and fainting spells. Another antitarget is the dopaminergic D2 receptor. The D2 receptor is a key antitarget associated with side effects such as extrapyramidal syndrome, and tardive dyskinesia. Another antitarget is the serotonin 5-HT2C receptor. The 5-HT2C receptor is a key antitarget associated with side effects related to weight gain and obesity. Another two antitargets are the serotonin 5-HT2B and 5-HT2A receptor which are the key antitargets associated with side effects related to valvular heart disease. Valvular heart disease caused by side effects related to the serotonin 5-HT2A and 5-HT2B receptors resulted in the withdrawal of the appetite suppressant fenfluramine (Pondimin) whose active metabolite norfenfluramine activates the cardiac 5-HT2B, as well as for the toxicity of two dopamine agonist drugs pergolide and cabergoline. Another antitarget is the muscarinic M1 receptor. The M1 receptor is a key antitarget for side effects associated with attention deficits, hallucinations and memory deficits.

### C. DEFINITIONS

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Antitargets

An “antitarget” or the like term refers to a receptor, enzyme or other biological target that is intervened by a drug, molecule, material, substance or drug candidate molecule, which causes undesirable or unwanted side effects. For example, an antitarget can be hERG, adrenergic alpha1A receptor, dopaminergic D2 receptor, serotonin 5-HT2C receptor, serotonin 5-HT2A receptor, serotonin 5-HT2B receptor or muscarinic M1 receptor or any combination thereof. An antitarget can also be can be a splicing variant or mutation of said receptors or antitargets. An antitarget can also comprise SEQ ID:1, SEQ ID NO:2, SEQ ID NO:3, SEQ ID NO:4, SEQ ID NO:6, SEQ ID NO:7, or SEQ ID NO.8 or any combination thereof.

5. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

6. Assaying the Response

“Assaying the response” or like terms means using a means to characterize the response. For example, if a molecule is brought into contact with a cell, a biosensor can be used to assay the response of the cell upon exposure to the molecule.

7. Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

8. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

9. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

10. Biosensor Signal

A “biosensor signal” or like terms refers to the signal of cells measured with a biosensor that is produced by the response of a cell upon stimulation.

11. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

12. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

13. Cell Panel

A “cell panel” or like terms is a panel which comprises at least two types of cells. The cells can be of any type or combination disclosed herein.

14. Cellular Response

A “cellular response” or like terms is any reaction by the cell to a stimulation.

15. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

16. Cellular Target

A “cellular target” or like terms is a biopolymer such as a protein or nucleic acid whose activity can be modified by an external stimulus. Cellular targets are most commonly proteins such as enzymes, kinases, ion channels, and receptors. Non-limiting examples of cellular target are hERG, adrenergic alpha1A receptor, dopaminergic D2 receptor, serotonin 5-HT2C receptor, serotonin 5-HT2A receptor, serotonin 5-HT2B receptor or muscarinic M1 receptor.

17. Characterizing

Characterizing or like terms refers to gathering information about any property of a substance, such as a ligand, molecule, marker, or cell, such as obtaining a profile for the ligand, molecule, marker, or cell.

18. Comprise

Throughout the description and claims of this specification, the word “comprise” and variations of the word, such as “comprising” and “comprises,” means “including but not limited to,” and is not intended to exclude, for example, other additives, components, integers or steps.

19. Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

20. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

21. Confluent Cell

A confluent cell or the like term refers to a cell monolayer with a surface coverage of at least 80%.

22. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

23. Common Cellular Background

“Common cellular background” or the like terms refers to a panel of cell types based on a common cell line. For example, HEK293 can be the cell line, then a panel of engineered HEK cells such as HEK-hERG, HEK-α1A, HEK-D2, HEK-5HT2A, HEK-5HT2C, HEK-5HT2B, or HEK-M1 or any combination thereof would be considered to be a part of a common cellular background with the HEK293 cell line because all cell types are based of HEK293 cells. Alternatively, Chinese hamster ovary (CHO-K1) can be the cell line, then a panel of engineered CHO cells such as CHO-hERG, CHO-α1A, CHO-D2, CHO-5HT2A, CHO-5HT2C, CHO-5HT2B, or CHO-M1 or any combination thereof would be considered to be a part of a common cellular background with the CHO-K1 cell line because all cell types are based of CHO-K1 cells.

24. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if a particular biological material, such as EGF, is disclosed EGF in its compound form is also disclosed.

25. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

26. Detect

Detect or like terms refer to an ability of the apparatus and methods of the disclosure to discover or sense a molecule- or a marker-induced cellular response and to distinguish the sensed responses for distinct molecules.

27. Direct Action (of a Candidate Antitarget Molecule)

A “direct action” or like terms is a result (of a candidate antitarget molecule”) acting independently on a cell.

28. DMR Signal

A “DMR signal” or like terms refers to the signal of cells measured with an optical biosensor that is produced by the response of a cell upon stimulation.

29. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

30. Drug Candidate Molecule

A drug candidate molecule or like terms is a test molecule which is or is going to be tested for its ability to function as a drug or a pharmacophore. This molecule may be considered as a lead molecule. A candidate antitarget molecule can also be a drug with potential risk(s) causing unwanted side effect(s) acting through the antitarget(s). A drug candidate molecule can act as a modulator to at least one antitarget.

31. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

32. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell Inhibit or forms of inhibit or like terms refers to reducing or suppressing.

33. Highly Confluent Cell

A confluent cell or the like term refers to a cell monolayer with a surface coverage of at least 90%.

34. In the Presence of the Molecule

“in the presence of the molecule” or like terms refers to the contact or exposure of the cultured cell with the molecule. The contact or exposure can be taken place before, or at the time, the stimulus is brought to contact with the cell.

35. Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

i. Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedance data point. It could be all of the data points associated with the profile curve.

ii. DMR Index

A “DMR index” or like terms is a biosensor index made up of a collection of DMR data.

iii. Safety Index

A “safety index” and the like terms refer to an index produced by data collected for a drug candidate molecule indicating the freedom from undesirable or unwanted side effects. For example, the safety index can indicate that a drug candidate molecule has many or severe side effects. The safety index can also indicate that a drug candidate molecule does not have any or only a few undesirable or unwanted side effects. The safety index of a drug candidate molecule can be compared to the safety index of a known drug with or without undesirable or unwanted side effects.

36. Known Molecule

A known molecule or like terms is a molecule with known pharmacological/biological/physiological/pathophysiological activity whose precise mode of action(s) may be known or unknown.

37. Known Modulator

A known modulator or like terms is a modulator where at least one of the targets is known with a known affinity. For example, a known modulator could be a PI3K inhibitor, a PKA inhibitor, a GPCR antagonist, a GPCR agonist, a RTK inhibitor, an epidermal growth factor receptor neutralizing antibody, or a phosphodiesterase inhibition, a PKC inhibitor or activator, etc.

38. Known Modulator Biosensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

39. Known Modulator DMR Index

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

40. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

41. Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

42. Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation). A hERG channel activator, such as mallotoxin, can be a marker for HEK-hERG cells, or HT29 cells, wherein hERG channels are stably expressed, or endogenously expressed in respective cells.

43. Marker Panel

A “marker panel” or like terms is a panel which comprises at least two markers. The markers can be for different pathways, the same pathway, different targets, or even the same targets. For example, mallotoxin can be used as a single marker for both HEK-hERG and HT29 cells. Thus for hERG channel modulator identification and classification, mallotoxin acts as an effective marker panel.

44. Marker Biosensor Index

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells. For hERG channel modulator identification and classification, the marker biosensor index includes the primary profiles of a drug candidate molecule across different cells (e.g., HEK293, HEK-hERG, HEK-alpha1A, HEK-5HT2A, HEK-5HT2B, HEK-5HT2C, HEK-D2, and HEK-M1 cells), and the modulation index of the drug candidate molecule against the DMR signals of the panel of markers, each is the agonist for respective target, in the cells. A drug candidate molecule that only results in a hERG-like DMR in HEK-hERG cell and causes the desensitization to the hERG marker mallotoxin-induced DMR signal is a hERG activator.

45. Marker DMR Index

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

46. Material

Material is the tangible part of something (chemical, biochemical, biological, or mixed) that goes into the makeup of a physical object.

47. Mimic

As used herein, “mimic” or like terms refers to performing one or more of the functions of a reference object. For example, a molecule mimic performs one or more of the functions of a molecule.

48. Mode of action

“Mode of action” or the like terms refer to the specific biochemical interaction through which a substance, molecule, drug, drug candidate molecule or material produces its pharmacological effect. A mode of action typically relates to the specific molecular targets of the substance, molecule, drug, drug candidate molecule or material, such as an enzyme or a receptor, e.g. hERG, adrenergic alpha1A receptor, dopaminergic D2 receptor, serotonin 5-HT2C receptor, serotonin 5-HT2A receptor, serotonin 5-HT2B receptor or muscarinic M1 receptor. The mode of action can be what target(s) are involved, which pathway(s) are involved and how are they influenced, it the substance, molecule, drug, drug candidate molecule or material an agonist or an antagonist towards the target.

49. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

50. Modulator

A modulator or like terms is a ligand that controls the activity of a cellular target. It is a signal modulating molecule binding to a cellular target, such as a target protein.

51. Modulate the Biosensor Signal of a Marker

“Modulate the biosensor signal or like terms is to cause changes of the biosensor signal or profile of a cell in response to stimulation with a marker.

52. Modulate the DMR Signal

“Modulate the DMR signal or like terms is to cause changes of the DMR signal or profile of a cell in response to stimulation with a marker.

53. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

54. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

55. Molecule Biosensor Index

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

56. Molecule DMR Index

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

57. Molecule Index

A “molecule index” or like terms is an index related to the molecule.

58. Molecule-Treated Cell

A molecule-treated cell or like terms is a cell that has been exposed to a molecule.

59. Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

60. Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target (e.g, hERG channel, hERG-associated signaling complex, PI3K, PKA, PKC, PKG, JAK2, MAPK, MEK2, or actin).

61. Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

62. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

63. Or

The word “or” or like terms as used herein means any one member of a particular list and also includes any combination of members of that list.

64. Parental Cell Line

A “parental cell line” or the like terms refer to a native cell line, either a transformed, or immortalized or primary cell. A parental cell line can be used as a control for distinguishing background-induced responses in a label free biosensor assay. For example, HEK293 is the parental cell line for HEK-hERG, HEK-α1A, HEK-D2, HEK-5HT2A, HEK-5HT2C, HEK-5HT2B or HEK-M1.

65. Profile

A profile or like terms refers to the data which is collected for a composition, such as a cell. A profile can be collected from a label free biosensor as described herein.

i. Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

ii. Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

iii. Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

66. Panel

A panel or like terms is a predetermined set of specimens (e.g., markers, or cells, or pathways). A panel can be produced from picking specimens from a library.

67. Positive Control

A “positive control” or like terms is a control that shows that the conditions for data collection can lead to data collection.

68. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

69. Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

70. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

71. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

72. Recombinantly Expressed Antitargets

“Recombinantly expressed antitargets” or “recombinantly expressed antitargets individually” or the like terms refer to a panel of cell types, such as a common cellular background, wherein the different cell types each express an antitarget. For example, a panel of cell types consisting of, for example, HEK-hERG, HEK-α1A, HEK-D2, HEK-5HT2A, HEK-5HT2B, HEK-5HT2C or HEK-M1 or any combination thereof would be considered to panel of cell types that recombinantly expressed antitargets.

73. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

74. “Robust DMR Signal”

A “robust DMR signal” or like terms is a DMR form of a “robust biosensor signal.”

75. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

76. Response

A response or like terms is any reaction to any stimulation.

77. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

78. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

79. Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Similarity analysis can be carried using Hierarchal Euclidean clustering analysis (Fang, Y. (2010) Label-free receptor assays. Drug Discovery Today: Technologies. DOI: 10.1016/j.ddtec.2010.05.001).

80. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

81. Substance

A substance or like terms is any physical object. A material is a substance. Molecules, ligands, markers, cells, proteins, and DNA can be considered substances. A machine or an article would be considered to be made of substances, rather than considered a substance themselves.

82. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

83. Test Molecule

A test molecule or like terms is a molecule which is used in a method to gain some information about the test molecule. A test molecule can be an unknown or a known molecule.

84. Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

85. Trigger

A trigger or like terms refers to the act of setting off or initiating an event, such as a response.

86. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

87. Unknown Molecule

An unknown molecule or like terms is a molecule with unknown biological/pharmacological/physiological/pathophysiological activity.

### D. BIOSENSORS

1. Acoustic Biosensors

Acoustic biosensors such as quartz crystal resonators utilize acoustic waves to characterize cellular responses. The acoustic waves are generally generated and received using piezoelectric. An acoustic biosensor is often designed to operate in a resonant type sensor configuration. In a typical setup, thin quartz discs are sandwiched between two gold electrodes. Application of an AC signal across electrodes leads to the excitation and oscillation of the crystal, which acts as a sensitive oscillator circuit. The output sensor signals are the resonance frequency and motional resistance. The resonance frequency is largely a linear function of total mass of adsorbed materials when the biosensor surface is rigid. Under liquid environments the acoustic sensor response is sensitive not only to the mass of bound molecules, but also to changes in viscoelastic properties and charge of the molecular complexes formed or live cells. By measuring the resonance frequency and the motion resistance of cells associated with the crystals, cellular processes including cell adhesion and cytotoxicity can be studied in real time.

2. Electrical Biosensors

Electrical biosensors employ impedance to characterize cellular responses including cell adhesion. In a typical setup, live cells are brought in contact with a biosensor surface wherein an integrated electrode array is embedded. A small AC pulse at a constant voltage and high frequency is used to generate an electric field between the electrodes, which are impeded by the presence of cells. The electric pulses are generated on site using the integrated electric circuit; and the electrical current through the circuit is followed with time. The resultant impedance is a measure of changes in the electrical conductivity of the cell layer. The cellular plasma membrane acts as an insulating agent forcing the current to flow between or beneath the cells, leading to quite robust changes in impedance. Impedance-based measurements have been applied to study a wide range of cellular events, including cell adhesion and spreading, cell micromotion, cell morphological changes, and cell death, and cell signaling.

3. Optical Biosensors

Optical biosensors primarily employ a surface-bound electromagnetic wave to characterize cellular responses. The surface-bound waves can be achieved either on gold substrates using either light excited surface plasmons (surface plasmon resonance, SPR) or on dielectric substrate using diffraction grating coupled waveguide mode resonances (resonance waveguide grating, RWG). For SPR, the readout is the resonance angle at which a minimal in intensity of reflected light occurs. Similarly, for RWG biosensor, the readout is the resonance angle or wavelength at which a maximum incoupling efficiency is achieved. The resonance angle or wavelength is a function of the local refractive index at or near the sensor surface. Unlike SPR, which is limited to a few of flow channels for assaying, RWG biosensors are amenable for high throughput screening (HTS) and cellular assays, due to recent advancements in instrumentation and assays. In a typical RWG, the cells are directly placed into a well of a microtiter plate in which a biosensor consisting of a material with high refractive index is embedded. Local changes in the refractive index lead to a dynamic mass redistribution (DMR) signal of live cells upon stimulation. These biosensors have been used to study diverse cellular processes including receptor biology, ligand pharmacology, and cell adhesion.

The present invention preferably uses resonant waveguide grating biosensors, such as Corning Epic® systems. Epic® system includes the commercially available wavelength integration system, or angular interrogation system or swept wavelength imaging system (Corning Inc., Corning, N.Y.). The commercial system consists of a temperature-control unit, an optical detection unit, with an on-board liquid handling unit with robotics, or an external liquid accessory system with robotics. The detection unit is centered on integrated fiber optics, and enables kinetic measures of cellular responses with a time interval of ˜7 or 15 sec. The compound solutions were introduced by using either the on-board liquid handling unit, or the external liquid accessory system; both of which use conventional liquid handling system.

4. Biosensors and Biosensor Assays

Label-free cell-based assays generally employ a biosensor to monitor molecule-induced responses in living cells. The molecule can be naturally occurring or synthetic, and can be a purified or unpurified mixture. A biosensor typically utilizes a transducer such as an optical, electrical, calorimetric, acoustic, magnetic, or like transducer, to convert a molecular recognition event or a molecule-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present methods can include, for example, optical biosensor systems such as surface plasmon resonance (SPR) and resonant waveguide grating (RWG) biosensors, resonant mirrors, ellipsometers, and electric biosensor systems such as bioimpedance systems.

i. SPR Biosensors and Systems

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface

ii. RWG Biosensors and Systems

An RWG biosensor can include, for example, a substrate (e.g., glass), a waveguide thin film with an embedded grating or periodic structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent wave to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured.

The resonance conditions are influenced by the cell layer (e.g., cell confluency, adhesion and status), which is in direct contact with the surface of the biosensor. When a ligand or an analyte interacts with a cellular target (e.g., a GPCR, an ion channel, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. Varying features of the systems can be automated, such as sample loading, and can be multiplexed, such as with a 96 or 386 well microtiter plate. Liquid handling is carried out by either on-board liquid handler, or an external liquid handling accessory. Specifically, molecule solutions are directly added or pipetted into the wells of a cell assay plate having cells cultured in the bottom of each well. The cell assay plate contains certain volume of assay buffer solution covering the cells. A simple mixing step by pipetting up and down certain times can also be incorporated into the molecule addition step.

iii. Electrical Biosensors and Systems

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

iv. High Spatial Resolution Biosensor Imaging Systems

Optical biosensor imaging systems, including SPR imaging systems, ellipsometry imaging systems, and RWG imaging systems, offer high spatial resolution, and can be used in embodiments of the disclosure. For example, SPR Imager® II (GWC Technologies Inc) uses prism-coupled SPR, and takes SPR measurements at a fixed angle of incidence, and collects the reflected light with a CCD camera. Changes on the surface are recorded as reflectivity changes. Thus, SPR imaging collects measurements for all elements of an array simultaneously.

A swept wavelength optical interrogation system based on RWG biosensor for imaging-based application can be employed. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of an image sensor naturally leads to an imaging based interrogation scheme. 2 dimensional label-free images can be obtained without moving parts.

Alternatively, angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds, or up to the whole 384well microplate to be simultaneously sampled at every 10 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved

v. Dynamic Mass Redistribution (DMR) Signals in Living Cells

The cellular response to stimulation through a cellular target can be encoded by the spatial and temporal dynamics of downstream signaling networks. For this reason, monitoring the integration of cell signaling in real time can provide physiologically relevant information that is useful in understanding cell biology and physiology.

Optical biosensors including resonant waveguide grating (RWG) biosensors, can detect an integrated cellular response related to dynamic redistribution of cellular matters, thus providing a non-invasive means for studying cell signaling. All optical biosensors are common in that they can measure changes in local refractive index at or very near the sensor surface. In principle, almost all optical biosensors are applicable for cell sensing, as they can employ an evanescent wave to characterize ligand-induced change in cells. The evanescent-wave is an electromagnetic field, created by the total internal reflection of light at a solution-surface interface, which typically extends a short distance (hundreds of nanometers) into the solution at a characteristic depth known as the penetration depth or sensing volume.

Recently, theoretical and mathematical models have been developed that describe the parameters and nature of optical signals measured in living cells in response to stimulation with ligands. These models, based on a 3-layer waveguide system in combination with known cellular biophysics, link the ligand-induced optical signals to specific cellular processes mediated through a receptor.

Because biosensors measure the average response of the cells located at the area illuminated by the incident light, a highly confluent layer of cells can be used to achieve optimal assay results. Due to the large dimension of the cells as compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) can be, to first order, directly proportional to the change in refractive index of the bottom portion of the cell layer:

ΔN=S(C)Δn_{c}

where S(C) is the sensitivity to the cell layer, and Δnc the ligand-induced change in local refractive index of the cell layer sensed by the biosensor. Because the refractive index of a given volume within a cell is largely determined by the concentrations of bio-molecules such as proteins, Δnc can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]$$

where ΔZ_{c} is the penetration depth into the cell layer, α the specific refraction increment (about 0.18/mL/g for proteins), zi the distance where the mass redistribution occurs, and d an imaginary thickness of a slice within the cell layer. Here the cell layer is divided into an equal-spaced slice in the vertical direction. The equation above indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it also is referred to as dynamic mass redistribution (DMR) signal.

5. Cells and Biosensors

Cells rely on multiple cellular pathways or machineries to process, encode and integrate the information they receive. Unlike the affinity analysis with optical biosensors that specifically measures the binding of analytes to a protein target, living cells are much more complex and dynamic.

To study cell signaling, cells can be brought into contact with the surface of a biosensor, which can be achieved through cell culture. These cultured cells can be attached onto the biosensor surface through three types of contacts: focal contacts, close contacts and extracellular matrix contacts, each with its own characteristic separation distance from the surface. As a result, the basal cell membranes are generally located away from the surface by ˜10-100 nm. For suspension cells, the cells can be brought in contact with the biosensor surface through either covalent coupling of cell surface receptors, or specific binding of cell surface receptors, or simply settlement by gravity force. For this reason, biosensors are able to sense the bottom portion of cells.

Cells, in many cases, exhibit surface-dependent adhesion and proliferation. In order to achieve robust cell assays, the biosensor surface can require a coating to enhance cell adhesion and proliferation. However, the surface properties can have a direct impact on cell biology. For example, surface-bound ligands can influence the response of cells, as can the mechanical compliance of a substrate material, which dictates how it will deform under forces applied by the cell. Due to differing culture conditions (time, serum concentration, confluency, etc.), the cellular status obtained can be distinct from one surface to another, and from one condition to another. Thus, special efforts to control cellular status can be necessary in order to develop biosensor-based cell assays.

Cells are dynamic objects with relatively large dimensions—typically in the range of tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

Under un-stimulated conditions cells generally produce an almost net-zero DMR response as examined with a RWG biosensor. This is partly because of the low spatial resolution of optical biosensors, as determined by the large size of the laser spot and the long propagation length of the coupled light. The size of the laser spot determines the size of the area studied—and usually only one analysis point can be tracked at a time. Thus, the biosensor typically measures an averaged response of a large population of cells located at the light incident area. Although cells undergo micromotion at the single cell level, the large populations of cells give rise to an average net-zero DMR response. Furthermore, intracellular macromolecules are highly organized and spatially restricted to appropriate sites in mammalian cells. The tightly controlled localization of proteins on and within cells determines specific cell functions and responses because the localization allows cells to regulate the specificity and efficiency of proteins interacting with their proper partners and to spatially separate protein activation and deactivation mechanisms. Because of this control, under un-stimulated conditions, the local mass density of cells within the sensing volume can reach an equilibrium state, thus leading to a net-zero optical response. In order to achieve a consistent optical response, the cells examined can be cultured under conventional culture conditions for a period of time such that most of the cells have just completed a single cycle of division.

Living cells have exquisite abilities to sense and respond to exogenous signals. Cell signaling was previously thought to function via linear routes where an environmental cue would trigger a linear chain of reactions resulting in a single well-defined response. However, research has shown that cellular responses to external stimuli are much more complicated. It has become apparent that the information that cells receive can be processed and encoded into complex temporal and spatial patterns of phosphorylation and topological relocation of signaling proteins. The spatial and temporal targeting of proteins to appropriate sites can be crucial to regulating the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution, when occurring within the sensing volume, can be followed directly in real time using optical biosensors

6. DMR Signal is a Physiological Response of Living Cells

Through comparison with conventional pharmacological approaches for studying receptor biology, it has been shown that when a ligand is specific to a receptor expressed in a cell system, the ligand-induced DMR signal is receptor-specific, dose-dependent and saturate-able. For a great number of G protein-coupled receptor (GPCR) ligands, the efficacies (measured by EC₅₀ values) are found to be almost identical to those measured using conventional methods. In addition, the DMR signals exhibit expected desensitization patterns, as desensitization and re-sensitization is common to all GPCRs. Furthermore, the DMR signal also maintains the fidelity of GPCR ligands, similar to those obtained using conventional technologies. In addition, the biosensor can distinguish full agonists, partial agonists, inverse agonists, antagonists, and allosteric modulators. Taken together, these findings indicate that the DMR is capable of monitoring physiological responses of living cells.

7. DMR Signals Contain Systems Cell Biology Information of Ligand-Receptor Pairs in Living Cells

The stimulation of cells with a ligand leads to a series of spatial and temporal events, non-limiting examples of which include ligand binding, receptor activation, protein recruitment, receptor internalization and recycling, second messenger alternation, cytoskeletal remodeling, gene expression, and cell adhesion changes. Because each cellular event has its own characteristics (e.g., kinetics, duration, amplitude, mass movement), and the biosensor is primarily sensitive to cellular events that involve mass redistribution within the sensing volume, these cellular events can contribute differently to the overall DMR signal. Chemical biology, cell biology and biophysical approaches can be used to elucidate the cellular mechanisms for a ligand-induced DMR signal. Recently, chemical biology, which directly uses chemicals for intervention in a specific cell signaling component, has been used to address biological questions. This is possible due to the identification of a great number of modulators that specifically control the activities of many different types of cellular targets. This approach has been adopted to map the signaling and its network interactions mediated through a receptor, including epidermal growth factor (EGF) receptor, and G_{q}- and Gₛ-coupled receptors.

8. DMR Signals Contain Systems Cell Pharmacology Information of a Ligand Acting on Living Cells.

Since the DMR signal is an integrated cellular response consisting of contributions of many cellular events involving dynamic redistribution of cellular matters within the bottom portion of cells, a ligand-induced biosensor signal, such as a DMR signal contains systems cell pharmacology information. It is known that GPCRs often display rich behaviors in cells, and that many ligands can induce operative bias to favor specific portions of the cell machinery and exhibit pathway-biased efficacies. Thus, it is highly possibly that a ligand can have multiple efficacies, depending on how cellular events downstream of the receptor are measured and used as readout(s) for the ligand pharmacology. It is difficult in practice for conventional cell assays, which are mostly pathway-biased and assay only a single signaling event, to systematically represent the signaling potentials of GPCR ligands. However, because label-free biosensors cellular assays do not require prior knowledge of cell signaling, and are pathway-unbiased and pathway-sensitive, these biosensor cellular assays are amenable to studying ligand-selective signaling as well as systems cell pharmacology of any ligands.

9. Biosensor Parameters

A label-free biosensor such as RWG biosensor or bioimpedance biosensor is able to follow in real time ligand-induced cellular response. The non-invasive and manipulation-free biosensor cellular assays do not require prior knowledge of cell signaling. The resultant biosensor signal contains high information relating to receptor signaling and ligand pharmacology. Multi-parameters can be extracted from the kinetic biosensor response of cells upon stimulation. These parameters include, but not limited to, the overall dynamics, phases, signal amplitudes, as well as kinetic parameters including the transition time from one phase to another, and the kinetics of each phase (see Fang, Y., and Ferrie, A. M. (2008) “label-free optical biosensor for ligand-directed functional selectivity acting on (32 adrenoceptor in living cells”. FEBS Lett. 582, 558-564; Fang, Y., et al., (2005) “Characteristics of dynamic mass redistribution of EGF receptor signaling in living cells measured with label free optical biosensors”. Anal. Chem., 77, 5720-5725; Fang, Y., et al., (2006) “Resonant waveguide grating biosensor for living cell sensing”. Biophys. J., 91, 1925-1940).

### E. METHODS

Disclosed herein are methods related to drug antitaget assessment using label-free cellular assays, particularly, label-free cellular pharmacology assays.

Disclosed herein are methods of assessing antitargets for a drug candidate molecule, comprising the steps of:

a. providing a common cellular background on a biosensor surface;

b. contacting the drug candidate molecule with the common cellular background;

c. analyzing the common cellular background; and

d. assessing the biosensor response of the drug candidate molecule on the antitarget.

Also disclosed herein are methods of screening candidate antitarget molecules, comprising the steps of:

a. providing a common cellular background on a biosensor surface;

b. contacting a candidate antitarget molecule with the common cellular background; and

c. analyzing the biosensor response of the candidate molecule on an antitarget in the common cellular background.

Also disclosed herein are methods of assessing antitargets for a drug candidate molecule, comprising the steps of:

a. providing an array of biosensors;

b. culturing a panel of cells onto the array of biosensors, wherein at least one type of cells recombinantly expressing an antitarget is plated onto at least one biosensor in the array, and all types of cells share a common cellular background;

c. contacting a drug candidate molecule with the panel of cells; and

d. analyzing the biosensor responses of the drug candidate molecule acting on the panel of cells.

Also disclosed herein are methods of assessing antitargets for a drug candidate molecule, comprising the steps of:

a. providing an array of biosensors;

b. culturing a cell onto the array of biosensors;

c. transfecting cells in the subset of biosensors in the array with a vector having an antitarget to recombinantly express the antitarget in the cells;

d. contacting a drug candidate molecule with the cells with and without the recombinantly expressed antitarget; and

e. analyzing the biosensor responses of the drug candidate molecule acting on the cells with and without the recombinantly expressed antitarget.

In one embodiment of the methods the common cellular background can be analyzed using a label-free biosensor. In some forms the label-free biosensor can be an Epic® system. Label-free biosensor cellular assays have been shown to be able to study the biology of receptors in live cells, and provide a functional readout (e.g., dynamic mass redistribution signal). The resultant DMR signal is an integrated cellular response, and follows the entire evolution of receptor activity. The real time kinetics enables classification of modes of action of modulators acting on the receptors and their regulatory proteins. Since label-free RWG biosensors measure an integrated and kinetic response of cells upon stimulation for the different cell types in the common cellular background with drug candidate molecules, the disclosed biosensor assays provide a new way to classify drug candidate molecules. In some forms a label-free biosensor for cellular assays can be a device consisting of three components—a biological component (i.e., live cells), a detector element, and a transducer associated with both components. Depending on the types of transducers used, label-free biosensors for whole cell sensing can be largely classified into three categories: acoustic, electrical and optical biosensors.

In another embodiment the common cellular background can be on an array of biosensor surfaces. In some forms the array of biosensor surfaces can be a 96well or 384well microplate. In some forms the 96well or 384well microplate can be a 96well Epic biosensor microplate or a 384well Epic biosensor microplate. In some forms the microplate can be coated with fibronectin.

The common cellular background enables the engineered cell types to be cultured on a single microtiter plate using one common culturing condition. Culture conditions are known to influence cellular context, including cell signaling. Thus, assaying cell types cultured using a common culturing condition can minimize unwanted environmental effects on label-free cellular profiles. Therefore, comparative analysis can be more effective and meaningful for a drug molecule acting on these cells. Since label-free cellular assays often lead to cellular background-dependent profiles induced via the activation of a receptor, a common cellular background can be important for effective determination of modes of action of a drug molecule acting on each antitarget receptor.

In another embodiment of the methods the seeding density of the cell in the common cellular background can be optimized. In some forms the seeding density for a 96well can be 30,000-40,000 cells per well. In some forms the seeding density for a 384well can be 5,000-12,000 cells per well.

In another embodiment cell types in the common cellular background can be maintained in a common medium. In some forms the medium can be MEM-Glutomax with 10% fetal bovine and 1% Penecillin/streptomycin.

In another embodiment of the methods the cell types in the common cellular background can be maintained in different mediums. In some forms each medium can be optimized for each cell type.

In another embodiment of the methods the cells in the common cellular background can be confluent. In some forms the common cellular background can be highly confluent.

In another embodiment of the methods the common cellular background can recombinantly express antitargets individually. In some forms the common cellular background can express any number of anti agents. In some forms the common cellular background can express 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 antitargets. In some forms the common cellular background can express 1, 2, 3, 4, 5, 6, 7, 8, 9, 10, 11 or 12 antitargets. In some forms the common cellular background can express 1, 2, 3, or 4 antitargets or 5, 6, 7, or 8 antitargets. In some forms the common cellular background can express 1, or 2 antitargets. In some forms the common cellular background can express 1, or 2, antitargets. In some forms the common cellular background can express 1 antitarget. In some forms the antitargets can be any antitarget. In some forms the antitarget can be hERG, adrenergic alpha1A receptor, dopaminergic D2 receptor, serotonin 5-HT2C receptor, serotonin 5HT-2A receptor, serotonin 5-HT2B receptor or muscarinic M1 receptor or any combination thereof. In some forms the antitarget can be a splicing variant or mutation of antitargets. In some forms the antitargets can comprise SEQ ID:1, SEQ ID NO:2, SEQ ID NO:3, SEQ ID NO:4, SEQ ID NO:6, SEQ ID NO:7, or SEQ ID NO:8, or any combination thereof.

In some embodiment, a native cell is used to culture onto all biosensors in an array of biosensors. A panel of vectors, at least one vector containing an antitarget gene, are used to transfect cells in a subset of biosensors in the array, such that these transfected cells recombinantly express the antitarget. Conventional physical approach-based (e.g., electroporation), transfection reagent based (e.g., FuGene or transfectamine) or viral based (e.g., Lentivirus) transfection can be used. Advances in recombinant DNA technologies have made effective and reproducible gene manipulation a routine in labs.

In another embodiment of the methods the common cellular background can comprise a parental cell line. In some forms the parent cell line can be HEK293, HeLa, Cos-7, NIH3T3, or CHO-K1 cells.

In another embodiment of the methods the common cellular background can comprise engineered cell types. In some forms the engineered cell types are based on the parental cell line. In some forms the parent cell line can be HEK293 or CHO-K1 cells. In some forms the engineered cell types can each express an antitarget. In some forms the engineered cell types can be HEK-hERG, HEK-α1A, HEK-D2, HEK-5HT2AB, HEK-5HT2A, HEK-5HT2C or HEK-M1 or any combination thereof.

In another embodiment the candidate antitarget molecule can be assayed at various concentrations. In some forms the candidate antitarget molecule can be assayed for the same cell type in the common cellular background at various concentrations. For example, a candidate antitarget molecule can be assayed for each cell type in the common cellular background in a dose response manner i.e. 0.01 μM, 0.05 μM, 0.10 μM, 0.5 μM, 1 μM, 5 μM, 10 μM, 50 μM, 100 μM or any combination thereof.

In another embodiment of the methods assessing the antitargets can comprise determining the mode of action of the drug candidate molecule on the antitargets. In some forms the mode of action can determined using an agonsim or antagonism assay or a combination thereof. In some forms an agonsim assay can be performed by recording cellular responses for 1 hour after adding the drug candidate molecule. In some forms an antagonism assay can be performed by prestimulating the cell types in the common cellular background for a period of time before stimulating the cell types with an agonist marker for its respective cell line. Since drug side effects due to the intervention of antitargets are related to specific modes of action of drug molecules acting on these antitargets, such mode of action assessment is crucial for antitarget assessment. For example, hERG blockage can cause long QT syndrome, wherein hERG activation can lead to short QT syndrome. In addition, stimulation of 5HT2A and 5-HT2B receptors is known to cause fibroblast mitogenesis, and the mitogenic effect has been linked to drug-induced valvular heart disease. The a1A adrenergic receptor modulates the relaxation of the vascular muscle tone and is thus important for blood pressure regulation. It has been suggested as an antitarget whose inhibition mediates cardiovascular side effects of many drug candidates, thereby causing orthostatic hypotension, dizziness, and fainting spells. In addition, inhibition of D2 receptor can lead to extrapyramidal syndrome and tardive dyskinesia. There is a relative segregation of outputs: the D1-mediated striatal output is directed preferentially to the globus pallidus, internal segment and substantia nigra, and pars reticulata, and the D2-mediated output is directed preferentially to the globus pallidus and external segment. By selectively blocking D2 receptors, long-term treatment with a conventional neuroleptic disrupts the normal, coordinated balance of D1- and D2-mediated striatal outputs. With long-term neuroleptic administration, endogenous dopamine is able to stimulate D1 receptors, whereas D2 receptors are occupied by neuroleptics, thus resulting in tardive dyskinesia. Furthermore, inhibition of 5-TH2C can result in weight gain and obesity, while inhibition of M1 receptor can result in attention deficits, hallucinations, and memory deficits.

In another embodiment of the methods assessing antitargets for the candidate antitarget molecule can comprise using the parental cell line as a control. In some forms the control can be a negative control. The parental cell line can measure background-induced responses which can be useful in label-free assays because the assays are largely non-specific. Since label-free cellular assays are considered to be multiplexing and non-specific, such a negative control is important to separate the antitarget-specific responses from the cellular background-induced responses.

In another embodiment the methods can further comprise generating a safety index for the drug candidate molecule. In some forms the safety index can be generated based on the collective assessment of all assayed antitargets. For example, if a drug candidate molecule is assayed against 6 or 7 antitargets then the results of all 6 or 7 antitargets could be combined in a safety index. In some forms the safety index can indicate how safe a drug candidate molecule is. In some forms the safety index can indicate how selective a drug candidate molecule is for the antitargets that the drug candidate molecule was assessed for. In some forms the safety index is compared to the safety index of known safe drug molecules. In some forms the safety index is compared to the safety index of known unsafe drug molecules. In some forms, the drug antitarget index (safety index) is generated using label-free cellular assays in combination with the antitarget-bearing cell panel. Traditionally a drug safety index is generated through the binding profile of a candidate antitarget molecule against a panel of receptors. The panel of receptors can include the antitargets described elsewhere herein. The functional consequences of drug binding to the antitargets are normally examined after the binding profile is acquired, typically using single readout based assays and/or animal models. This process is both time consuming and ineffective, because a drug molecule can display functional selectivity. For example, a drug molecule that binds to the alpha1A receptor and behaves as an alpha1A antagonist in a classical second message assay (e.g., cAMP or Ca2+) may not cause any cardiovascular side effect, such as orthostatic hypotension, dizziness or fainting spell. This is because a second message assay only represents a single cellular event downstream from the alpha1A receptor signaling, however, the drug molecule can be a functional selective agonism in pathways or cellular processes other than the second message level. In one embodiment of the methods the label-free cellular assays can be pathway-unbiased but pathway-sensitive. Thus, the label-free cellular assays can provide high texture in detecting the functional sequence of drug molecules.

In another embodiment the methods can further comprise determining if a drug candidate molecule is safe. In some forms determining if the drug candidate molecule is safe can comprise determining the mode of action of the drug candidate molecule on the antitargets. The mode of action of the drug candidate molecule on the antitarget can determine if the drug candidate molecule can cause unwanted side effect if administered to a subject. In some forms the mode of action can be determined using an agonsim or antagonism assay or a combination thereof.

In another embodiment the methods further comprise determining the specific mode of the drug candidate molecule. The assessment of specific modes of action for drug candidate molecules is important because it relates to the effect the drug candidate molecule has on the antitarget and, thus, can be crucial for antitarget assessment. For example, different drug candidate molecules can influence the antitarget differently leading to different side effects, i.e. hERG blockage can cause long QT syndrome, while hERG activation can lead to short QT syndrome. Furthermore, the modulation of the alpha1A adrenergic receptor, dopaminergic D2 receptor, serotonin 5-HT2C receptor, serotonin 5-HT2B receptor, and muscarinic M1 receptor lead to the unwanted side effects associated with these receptors. In some embodiments of the methods disclosed herein a cellular function assay detects the mode of action of a drug candidate molecule acting on each of these antitargets in living cells.

In one embodiment the methods relate to a panel of important antitargets of candidate antitarget molecules. In some forms the important antitargets can be human ether-à-go-go-related gene (hERG) channel (e.g., hERG1), alpha1A adrenergic receptor, dopaminergic D2 receptor, serotonin 5HT-2A, serotonin 5-HT2C receptor, serotonin 5-HT2B receptor, or muscarinic M1 receptor or any combination thereof. In another embodiment, the antitargets can be splicing variants or mutants of the receptors disclosed elsewhere herein.

One embodiment of the methods screens and identifies drug candidate molecules with a desired clinical safety profile. For example, a drug candidate molecule would have a desired clinical safety profile if it does not adversely affect any antitarget. However, a drug candidate molecule can still have a desired clinical safety profile even if it only adversely affect an antitarget at high doses.

### F. EXAMPLES

### 1. Example 1

### Culture of 8 Different Cell Types in a 96well Biosensor Mircoplate

i. Material

All cell culture reagents were purchased from Invitrogen GIBCO cell culture products.

ii. Methods

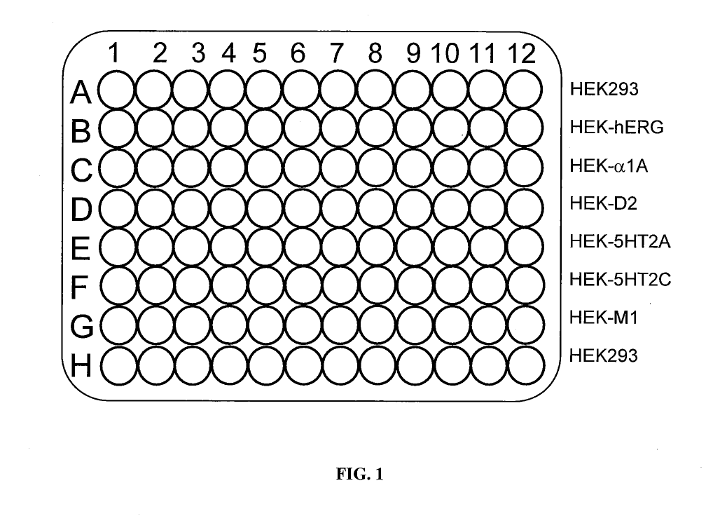

8 different cell types were cultured in a 96well Epic biosensor microplate (e.g., Epic microplate fibronectin coated, Corning Inc.) as described in FIG. 1. The 8 cell types include the parental human embryonic kidney HEK293 cell as a negative control, and 7 engineered HEK cell lines. Each HEK cell line expresses a specific receptor (e.g., hERG, alpha1A adrenergic receptor, dopamine D2 receptor, 5HT-2A, 5HT-2B, 5HT-2C and muscarinic M1 receptor, respectively). Chinese hamster ovary (CHO-K1), NIH3T3, or Cos-7 cells can also be engineered similarly to the HEK cell types as described above to be used as the common cellular background.

These engineered cell lines can be purchased from commercial vendors (e.g., American Tissue Cell Culture (ATCC), Invitrogen, DiscoverRx, Euroscreen, PerkinElmer) or they can be made. Since all cells share a common cellular background (e.g., HEK293) they can be cultured on a single biosensor microplate using a common culturing conditions. The cell seeding density can be optimized (and is typically between 30-40K cells per well in a 96well Epic biosensor microplate, or between 5-12K cells per well in a 384well Epic biosensor microplate).

The cell types can be maintained in a single medium or in different mediums, each medium being optimized for each cell type. For example, all cells can be maintained in MEM-GlutoMax with 10% fetal bovine serum and 1% Pennicillinistreptomycin. The cell types were subcultured 1-2 times per week and cell passage less than 15 was used for all experiments. All cell types were cultured in 5% carbon dioxide in a standard cell culture incubator. Alternatively, a batch of cells can be pre-made and frozen stored. Once thawed, these cells can be directly plated in the biosensor microplates at desired seeding density. Once cultured overnight, these cells can be assayed using the biosensor cellular assays.

The cell types became confluent after being cultured overnight. The cell types were then washed twice on a BioTek ELx405 Select washer with Hank's Balanced Salt Solution (HBSS) containing 20 mM Hepes (1×HBSS). 1×HBB is the common buffer used in label-free biosensor cellular assays. After washing, the cell types on the biosensor plate are incubated in a HBSS buffer at 28° C. inside the Epic system for one hour. The Epic® wavelength interrogation system (Corning Inc., Corning, N.Y.) consists of a temperature-control unit, an optical detection unit, and an on-board liquid handling unit with robotics. The detection unit is centered on integrated fiber optics, and enables kinetic measures of cellular responses with a time interval of ˜15 sec. For the agonism assay, a 2-min baseline is initiated, followed by addition of 10 μl test drug solutions (5×). The cell responses for each cell type are recorded continuously for one hour. For the antagonism assay, the cells are prestimulated with a test drug molecule for a given period of time (typically 30 min to 1 hour), and then stimulated with corresponding agonist marker panel, each marker for its respective cell line. Examples are mallotoxin or other hERG activator as the marker for hERG, oxymetazoline and other alpha1A agonists as the marker for alpha1A adrenergic receptor, dopamine and other D2 agonists as the marker for dopamine D2 receptors, serotonin and other 5HT-2A, 5-HT2B, or 5-TH2C agonists as the marker for 5-HT2A, 5-HT2B, or 5-HT2C receptors, and acetylcholine and other M1 agonists as the markers for muscarinic M1 receptor. Full agonists for respective antitarget receptors are preferred. For the antagonism assays, each marker is preferred to be assayed at EC50, EC80, 1×EC100 or 5×EC100. The EC50 or EC80 or EC100 for each marker is preferred to be determined using label-free cellular assays in respective cell lines. Since biosensor 96well or 384well or any other format microtiter plates can be considered as an array of biosensors, the footprint or assay layout for all cells within a given plate can be flexible, depending on the number of drug candidate molecules to be assessed, and the information content to be obtained. Additional cell lines expressing other antitarget receptors can also be included to achieve broad spectrum of antitarget assessment, and in general, drug safety assessment.

### G. SEQUENCES

The protein sequence of hERG1a (SEQ ID NO:1) (NCBI Reference Sequence: NP 000229.1) is:

MPVRRGHVAPQNTFLDTIIRKFEGQSRKFIIANARVENCAVIYCNDGFCE
LCGYSRAEVMQRPCTCDFLHGPRTQRRAAAQIAQALLGAEERKVEIAFYR
KDGSCFLCLVDVVPVKNEDGAVIMFILNFEVVMEKDMVGSPAHDTNHRGP
PTSWLAPGRAKTFRLKLPALLALTARESSVRSGGAGGAGAPGAVVVDVDL
TPAAPSSESLALDEVTAMDNHVAGLGPAEERRALVGPGSPPRSAPGQLPS
PRAHSLNPDASGSSCSLARTRSRESCASVRRASSADDIEAMRAGVLPPPP
RHASTGAMHPLRSGLLNSTSDSDLVRYRTISKIPQITLNFVDLKGDPFLA
SPTSDREIIAPKIKERTHNVTEKVTQVLSLGADVLPEYKLQAPRIHRWTI
LHYSPFKAVWDWLILLLVIYTAVFTPYSAAFLLKETEEGPPATECGYACQ
PLAVVDLIVDIMFIVDILINFRTTYVNANEEVVSHPGRIAVHYFKGWFLI
DMVAAIPFDLLIFGSGSEELIGLLKTARLLRLVRVARKLDRYSEYGAAVL
FLLMCTFALIAHWLACIWYAIGNMEQPHMDSRIGWLHNLGDQIGKPYNSS
GLGGPSIKDKYVTALYFTFSSLTSVGFGNVSPNTNSEKIFSICVMLIGSL
MYASIFGNVSAIIQRLYSGTARYHTQMLRVREFIRFHQIPNPLRQRLEEY
FQHAWSYTNGIDMNAVLKGFPECLQADICLHLNRSLLQHCKPFRGATKGC
LRALAMKFKTTHAPPGDTLVHAGDLLTALYFISRGSIEILRGDVVVAILG
KNDIFGEPLNLYARPGKSNGDVRALTYCDLHKIHIRDDLLEVLDMYPEFS
DHFWSSLEITFNLRDTNMIPGSPGSTELEGGFSRQRKRKLSFRRRTDKDT
EQPGEVSALGPGRAGAGPSSRGRPGGPWGESPSSGPSSPESSEDEGPGRS
SSPLRLVPFSSPRPPGEPPGGEPLMEDCEKSSDTCNPLSGAFSGVSNIFS
FWGDSRGRQYQELPRCPAPTPSLLNIPLSSPGRRPRGDVESRLDALQRQL
NRLETRLSADMATVLQLLQRQMTLVPPAYSAVTTPGPGPTSTSPLLPVSP
LPTLTLDSLSQVSQFMACEELPPGAPELPQEGPTRRLSLPGQLGALTSQP
LHRHGSDPGS

The protein sequence of hERG1b (SEQ ID NO:2) (NCBI Reference Sequence: NP 742053.1) is:

MPVRRGHVAPQNTFLDTIIRKFEGQSRKFIIANARVENCAVIYCNDGFCE
LCGYSRAEVMQRPCTCDFLHGPRTQRRAAAQIAQALLGAEERKVEIAFYR
KDGSCFLCLVDVVPVKNEDGAVIMFILNFEVVMEKDMVGSPAHDTNHRGP
PTSWLAPGRAKTFRLKLPALLALTARESSVRSGGAGGAGAPGAVVVDVDL
TPAAPSSESLALDEVTAMDNHVAGLGPAEERRALVGPGSPPRSAPGQLPS
PRAHSLNPDASGSSCSLARTRSRESCASVRRASSADDIEAMRAGVLPPPP
RHASTGAMHPLRSGLLNSTSDSDLVRYRTISKIPQITLNFVDLKGDPFLA
SPTSDREIIAPKIKERTHNVTEKVTQVLSLGADVLPEYKLQAPRIHRWTI
LHYSPFKAVWDWLILLLVIYTAVFTPYSAAFLLKETEEGPPATECGYACQ
PLAVVDLIVDIMFIVDILINFRTTYVNANEEVVSHPGRIAVHYFKGWFLI
DMVAAIPFDLLIFGSGSEELIGLLKTARLLRLVRVARKLDRYSEYGAAVL
FLLMCTFALIAHWLACIWYAIGNMEQPHMDSRIGWLHNLGDQIGKPYNSS
GLGGPSIKDKYVTALYFTESSLTSVGFGNVSPNTNSEKIFSICVMLIGSL
MYASIFGNVSAIIQRLYSGTARYHTQMLRVREFIRFHQIPNPLRQRLEEY
FQHAWSYTNGIDMNAVLKGFPECLQADICLHLNRSLLQHCKPFRGATKGC
LRALAMKFKTTHAPPGDTLVHAGDLLTALYFISRGSIEILRGDVVVAILG
MGWGAGTGLEMPSAASRGASLLNMQSLGLWTWDCLQGHWAPLIHLNSGPP
SGAMERSPTWGEAAELWGSHILLPFRIRHKQTLFASLK

The protein sequence of alpha1A adrenergic receptor (SEQ ID NO:3) (UniProtKB/Swiss-Prot P35348-1) is:

MVFLSGNASDSSNCTQPPAPVNISKAILLGVILGGLILFGVLGNILVILS
VACHRHLHSVTHYYIVNLAVADLLLTSTVLPFSAIFEVLGYWAFGRVFCN
IWAAVDVLCCTASIMGLCIISIDRYIGVSYPLRYPTIVTQRRGLMALLCV
WALSLVISIGPLFGWRQPAPEDETICQINEEPGYVLFSALGSFYLPLAII
LVMYCRVYVVAKRESRGLKSGLKTDKSDSEQVTLRIHRKNAPAGGSGMAS
AKTKTHFSVRLLKFSREKKAAKTLGIVVGCFVLCWLPFFLVMPIGSFFPD
FKPSETVFKIVFWLGYLNSCINPITYPCSSQEFKKAFQNVLRIQCLCRKQ
SSKHALGYTLHPPSQAVEGQHKDMVRIPVGSRETFYRISKTDGVCEWKFF
SSMPRGSARITVSKDQSSCTTARVRSKSFLQVCCCVGPSTPSLDKNHQVP
TIKVHTISLSENGEEV

The protein sequence of human dopamine D2 receptor (SEQ ID NO:4) (UniProtKB/Swiss-Prot P14416-1) is:

MDPLNLSWYDDDLERQNWSRPFNGSDGKADRPHYNYYATLLTLLIAVIVF
GNVLVCMAVSREKALQTTTNYLIVSLAVADLLVATLVMPWVVYLEVVGEW
KFSRIHCDIFVTLDVMMCTASILNLCAISIDRYTAVAMPMLYNTRYSSKR
RVTVMISIVWVLSFTISCPLLFGLNNADQNECIIANPAFVVYSSIVSFYV
PFIVTLLVYIKIYIVLRRRRKRVNTKRSSRAFRAHLRAPLKGNCTHPEDM
KLCTVIMKSNGSFPVNRRRVEAARRAQELEMEMLSSTSPPERTRYSPIPP
SHHQLTLPDPSHHGLHSTPDSPAKPEKNGHAKDHPKIAKIFEIQTMPNGK
TRTSLKTMSRRKLSQQKEKKATQMLAIVLGVFIICWLPFFITHILNIHCD
CNIPPVLYSAFTWLGYVNSAVNPIIYTTFNIEFRKAFLKILHC

The protein sequence of human 5HT-2A receptor (SEQ ID NO:5) (UniProtKB/Swiss-Prot P28223) is:

MDILCEENTSLSSTTNSLMQLNDDTRLYSNDFNSGEANTSDAFNWTVDSE
NRTNLSCEGCLSPSCLSLLHLQEKNWSALLTAVVIILTIAGNILVIMAVS
LEKKLQNATNYFLMSLAIADMLLGFLVMPVSMLTILYGYRWPLPSKLCAV
WIYLDVLFSTASIMHLCAISLDRYVAIQNPIHHSRFNSRTKAFLKIIAVW
TISVGISMPIPVFGLQDDSKVFKEGSCLLADDNFVLIGSFVSFFIPLTIM
VITYFLTIKSLQKEATLCVSDLGTRAKLASFSFLPQSSLSSEKLFQRSIH
REPGSYTGRRTMQSISNEQKACKVLGIVFFLFVVMWCPFFITNIMAVICK
ESCNEDVIGALLNVFVWIGYLSSAVNPLVYTLFNKTYRSAFSRYIQCQYK
ENKKPLQLILVNTIPALAYKSSQLQMGQKKNSKQDAKTTDNDCSMVALGK
QHSEEASKDNSDGVNEKVSCV

The protein sequence of human 5HT-2C receptor (SEQ ID NO:6) (UniProtKB/Swiss-Prot P28335) is:

MVNLRNAVHSFLVHLIGLLVWQCDISVSPVAAIVTDIFNTSDGGRFKFPD
GVQNWPALSIVIIIIMTIGGNILVIMAVSMEKKLHNATNYFLMSLAIADM
LVGLLVMPLSLLAILYDYVWPLPRYLCPVWISLDVLFSTASIMHLCAISL
DRYVAIRNPIEHSRFNSRTKAIMKIAIVWAISIGVSVPIPVIGLRDEEKV
FVNNTTCVLNDPNFVLIGSFVAFFIPLTIMVITYCLTIYVLRRQALMLLH
GHTEEPPGLSLDFLKCCKRNTAEEENSANPNQDQNARRRKKKERRPRGTM
QAINNERKASKVLGIVFFVFLIMWCPFFITNILSVLCEKSCNQKLMEKLL
NVFVWIGYVCSGINPLVYTLFNKIYRRAFSNYLRCNYKVEKKPPVRQIPR
VAATALSGRELNVNIYRHTNEPVIEKASDNEPGIEMQVENLELPVNPSSV
VSERISSV

The protein sequence of human Muscarinic acetylcholine receptor M1 receptor (SEQ ID NO:7) (UniProtKB/Swiss-Prot P11229) is:

MNTSAPPAVSPNITVLAPGKGPWQVAFIGITTGLLSLATVTGNLLVLISF
KVNTELKTVNNYFLLSLACADLIIGTFSMNLYTTYLLMGHWALGTLACDL
WLALDYVASNASVMNLLLISFDRYFSVTRPLSYRAKRTPRRAALMIGLAW
LVSFVLWAPAILFWQYLVGERTVLAGQCYIQFLSQPIITFGTAMAAFYLP
VTVMCTLYWRIYRETENRARELAALQGSETPGKGGGSSSSSERSQPGAEG
SPETPPGRCCRCCRAPRLLQAYSWKEEEEEDEGSMESLTSSEGEEPGSEV
VIKMPMVDPEAQAPTKQPPRSSPNTVKRPTKKGRDRAGKGQKPRGKEQLA
KRKTFSLVKEKKAARTLSAILLAFILTWTPYNIMVLVSTFCKDCVPETLW
ELGYWLCYVNSTINPMCYALCNKAFRDTFRLLLLCRWDKRRWRKIPKRPG
SVHRTPSRQC

The protein sequence of human 5HT-2B receptor (SEQ ID NO:8) (NP_{—}000858) is:

| | |
|---|---|
| 1 | malsyrvsel qstipehilq stfvhvissn wsglqtesip eemkqiveeq gnklhwaall |
| 61 | ilmviiptig gntivilavs lekklqyatn yflmslavad llvglfvmpi alltimfeam |
| 121 | wplplvlcpa wlfldvlfst asimhlcais vdryiaikkp iqanqynsra tafikitvvw |
| 181 | lisigiaipv pikgietdvd npnnitcvlt kerfgdfmlf gslaafftpl aimivtyflt |
| 241 | ihalqkkayl vknkppqrlt wltvstvfqr detpcsspek vamldgsrkd kalpnsgdet |
| 301 | lmrrtstigk ksvqtisneq raskvlgivf flfllmwcpf fitnitivlc dscnqttlqm |
| 361 | lleifvwigy vssgvnplvy tlfnktfrda fgryitcnyr atksvktlrk rsskiyfrnp |
| 421 | maenskffkk hgirnginpa myqspmrlrs stiqsssiil ldtllltene gdkteeqvsy |
| 481 | v |

### H. REFERENCES

1. U.S. application Ser. No. 12/623,693. Fang, Y., Ferrie, A. M., Lahiri, J., and Tran, E. “Methods for Characterizing Molecules”, Filed Nov. 23, 2009
2. U.S. application Ser. No. 12/623,708. Fang, Y., Ferrie, A. M., Lahiri, J., and Tran, E. “Methods of creating an index”, filed Nov. 23, 2009

We claim:

1. A method of assessing antitargets for a drug candidate molecule, comprising the steps of
a. providing a common cellular background on a biosensor surface;
b. contacting the drug candidate molecule with the common cellular background;
c. analyzing the common cellular background; and
d. assessing the biosensor response of the drug candidate molecule on the antitarget.

2. The method of claim 1, wherein the common cellular background is analyzed using a label-free biosensor.

3. The method of claim 1, wherein the common cellular background recombinantly expresses an antitarget individually.

4. The method of claim 1, wherein the common cellular background expresses 1, 2, 3, 4, 5, 6, 7, 8, 9, 10, 11, 12, 13, 14, 15 or 20 antitargets.

5. The method of claim 1, wherein the common cellular background express hERG, adrenergic alpha1A receptor, dopaminergic D2 receptor, serotonin 5-HT2C receptor, serotonin 5-HT2A receptor, serotonin 5-HT2B receptor or muscarinic M1 receptor or any combination thereof.

6. The method of claim 1, wherein assessing antitargets for the drug candidate molecule comprises using the parental cell line as a negative control.

7. The method of claim 1, further comprising generating a safety index for the drug candidate molecule.

8. The method of claim 1, wherein the common cellular background comprises HEK293 or CHO-K1 cells.

9. A method of screening candidate antitarget molecules, comprising the steps of:
a. providing a common cellular background on a biosensor surface;
b. contacting a candidate antitarget molecule with the common cellular background; and
c. analyzing the biosensor response of the candidate antitarget molecule on an antitarget in the common cellular background.

10. The method of claim 9, wherein the common cellular background is analyzed using a label-free biosensor.

11. The method of claim 9, wherein the common cellular background recombinantly express an antitarget individually.

12. The method of claim 9, wherein the common cellular background express 1, 2, 3, 4, 5, 6, 7, 8, 9, 10, 11, 12, 13, 14, 15 or 20 antitargets.

13. The method of claim 9, further comprising determining if the candidate antitarget molecule is safe.

14. The method of claim 9, wherein the common cellular background express hERG, adrenergic alpha1A receptor, dopaminergic D2 receptor, serotonin 5-HT2C receptor, serotonin 5-HT2A receptor, serotonin 5-HT2B receptor or muscarinic M1 receptor or any combination thereof.

15. The method of claim 9, wherein determining if the candidate antitarget molecule is safe comprises generating safety index for the candidate antitarget molecule.

16. The method of claim 9, wherein the common cellular background comprise HEK293 or CHO-K1 cells.

17. A method of assessing antitargets for a drug candidate molecule, comprising the steps of:
a. providing an array of biosensors;
b. culturing a panel of cells onto the array of biosensors, wherein at least one type of cells recombinantly expressing an antitarget is plated onto at least one biosensor in the array, and all types of cells share a common cellular background;
c. contacting a drug candidate molecule with the panel of cells; and
d. analyzing the biosensor responses of the drug candidate molecule acting on the panel of cells.

18. The method of claim 17, wherein the array of biosensors is in microtiter plate format.

19. A method of assessing antitargets for a drug candidate molecule, comprising the steps of:
a. providing an array of biosensors;
b. culturing a cell onto the array of biosensors;
c. transfecting cells in the subset of biosensors in the array with a vector having an antitarget to recombinantly express the antitarget in the cells;
d. contacting a drug candidate molecule with the cells with and without the recombinantly expressed antitarget; and
e. analyzing the biosensor responses of the drug candidate molecule acting on the cells with and without the recombinantly expressed antitarget.

20. The method of claim 19, wherein the array of biosensors is in a microtiter plate format.


SEQUENCE LISTING (8 sequences)

SEQ ID NO: 1 - protein, 1159 aa, Homo sapiens
  PEPTIDE (1)..(1159)
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 2 - protein, 888 aa, Homo sapiens
  PEPTIDE (1)..(888)
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGMGWGAGTGLEMPSAASRGASLLNMQSLGLWTWDCLQGHWAPLIHLNSGPPSGAMERSPTWGEAAELWGSHILLPFRIRHKQTLFASLK

SEQ ID NO: 3 - protein, 466 aa, Homo sapiens
  PEPTIDE (1)..(466)
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLCRKQSSKHALGYTLHPPSQAVEGQHKDMVRIPVGSRETFYRISKTDGVCEWKFFSSMPRGSARITVSKDQSSCTTARVRSKSFLQVCCCVGPSTPSLDKNHQVPTIKVHTISLSENGEEV

SEQ ID NO: 4 - protein, 443 aa, Homo sapiens
  PEPTIDE (1)..(443)
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 5 - protein, 471 aa, Homo sapiens
  PEPTIDE (1)..(471)
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 6 - protein, 458 aa, Homo sapiens
  PEPTIDE (1)..(458)
MVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 7 - protein, 460 aa, Homo sapiens
  PEPTIDE (1)..(460)
MNTSAPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPETPPGRCCRCCRAPRLLQAYSWKEEEEEDEGSMESLTSSEGEEPGSEVVIKMPMVDPEAQAPTKQPPRSSPNTVKRPTKKGRDRAGKGQKPRGKEQLAKRKTFSLVKEKKAARTLSAILLAFILTWTPYNIMVLVSTFCKDCVPETLWELGYWLCYVNSTINPMCYALCNKAFRDTFRLLLLCRWDKRRWRKIPKRPGSVHRTPSRQC

SEQ ID NO: 8 - protein, 481 aa, Homo sapiens
  PEPTIDE (1)..(481)
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEEQVSYV